SEQUENCE LISTING (1 sequence)

SEQ ID NO: 1 - protein, 559 aa, Leishmania donovani
MLRIAVSTEDLTDPAYHCARVGQRIKHALGRFAICTAEDILTDEKRDILVKHLIPQALQLHTERLKVRQVQDKWKVTGMGDDVCSDFKVPPAHITDGLSNTDFVMYVASVPSEGDVLAWATTCQVFSDGHPAVGVVNIPAANIASRYDQLVTRVVTHEMAHALGFSVGFFEGARILENISNVRHKDFDVPVINQDLPEFFDAAEHWPMCVTISEIRDQSNCGSCWAIAAVEAISDRYCTLGGVPDRRISTSNLLSCCFICGFGCYGGIPTMAWLWWVWVGITTEVCQPYPFGPCSHHGNSDKGSASFKYAWVLDKLKAERERGITIDIALWKFESPKSVFTIIDAPGHRDFIKNMITGTSQADAAILMIDSTHGGFEAGISKDGQTREHALLAFTLGVKQMVVCCNKMDDKTVTYAQSRYDEISKEVGAYLKRVGYNPEKVRFIPISGWQGDNMIERSDNMPWYKGPTLLDALDMLEPPVRPVDKPLRLPLQDVYKIGGIGTVPVGRVETGIMKPGDVVTFAPANVTTEVKSIEMHHEQLAEAQPGDNVGFNVHHHHHH

(19) United States
(12) Patent Application Publication
(10) Pub. No.: US 2026/0284167 A1
(43) Pub. Date: Sep. 24, 2026

(54) LIPOSOMAL FORMULATION FOR TREATMENT OF VISCERAL LEISHMANIASIS

(71) Applicant: Council of Scientific & Industrial Research, New Delhi (IN)

(72) Inventors: Nahid ALI, Kolkata (IN); Nicky DIDWANIA, Kolkata (IN); Mohd KAMRAN, Boston, MA (US); Abdus SABUR, Raiganj (IN); Sarfaraz Ahmad EJAZI, College Park, MD (US)

(21) Appl. No.: 19/476,508

(22) PCT Filed: Apr. 10, 2024
(86) PCT No.: PCT/IN2024/050371

§ 371 Date: Oct. 17, 2025

(30) Foreign Application Priority Data
Apr. 20, 2023 (IN) 202311028946

(51) Int. Cl. A61K 39/008; A61K 9/00; A61K 9/1277; A61K 39/00; A61P 33/02; C07K 14/44; C12N 15/63; G01N 33/53

(52) U.S. Cl. CPC A61K 39/008; A61K 9/0019; A61K 9/1277; A61P 33/02; C07K 14/44; C12N 15/63; G01N 33/53; A61K 2039/54; A61K 2039/545; A61K 2039/55555; C07K 2319/00; G01N 2800/26

(57) ABSTRACT

Antigens from multiple sources is a promising strategy to effectively stimulate all immuno-phenotypic sections of a diverse population. Therefore, combination of multiple protective antigens is a rationale strategy to boost immunogenicity of the vaccine designed for immunization in heterogeneous population as well as to use in effective diagnosis in all endemic areas. The strategy to combine antigens isisis to construct a fusion chimeric multivalent antigen. The present invention relates to the diagnostic and vaccine potential of a recombinant multiantigenic T cell epitope enriched fusion protein comprising of three Leishmania donovani proteins, glycoprotein 63 (GP63), elongation factor 1 α (EF1-α) and cysteine protease C (CPC) against visceral leishmaniasis. The said invention in particular relates to designing, cloning and purification of a novel T cell enriched multiantigenic recombinant protein for detection of Leishmania infection in the form of antigen-specific antibodies in the biological samples such as serum and urine.

### TECHNICAL FIELD OF THE INVENTION

The present invention relates to liposomal formulation for treatment of visceral leishmaniasis. More particularly it relates to a recombinant multiantigenic T cell epitope enriched fusion protein comprising of three Leishmania donovani proteins, glycoprotein 63 (GP63), elongation factor 1 α (EF1-α) and cysteine protease C (CPC) against visceral leishmaniasis.

The present invention in particular relates to designing, cloning and purification of a novel T cell enriched multiantigenic recombinant protein.

The present invention in particular relates to detection of Leishmania infection in the form of antigen-specific antibodies in the biological samples such as serum and urine.

More particularly, the present invention relates to the protective efficacy of the antigen in BALB/c mice against experimental visceral leishmaniasis.

### BACKGROUND OF THE INVENTION

There is no vaccine commercially available for visceral leishmaniasis (VL). However, several attempts have been made in previous studies. Live vaccines using attenuated Leishmania parasites have been shown to be effective in multiple experimental studies (Ismail, Kaul et al. 2017). However, they have been restricted from clinical application due to safety concerns. On the other hand defined subunit vaccines though considered safe are limited by their poor immunogenicity. Several studies with protein based vaccines have been carried out against VL (Das and Ali 2012) (Didwania, Shadab et al. 2017). Among them, soluble L. donovani membrane antigens (SLA) have been reported to confer almost complete protection and excellent immunotherapy against experimental VL (Bhowmick, Ravindran et al. 2007). The most immunodominant components of SLA, identified as glycoprotein 63 (GP63) and elongation factor 1-alpha (EF1-α), both in native and recombinant form were also found to induce prophylactic immunity against VL (Bhowmick, Ravindran et al. 2008) (Mazumder, Maji et al. 2011) (Sabur, Bhowmick et al. 2018). Additionally, comparisons of the family of the cysteine proteases (CPs) for prophylactic efficacy against VL revealed CPC to be most protective. Therefore, in our earlier studies we have established that CPC along with GP63 and EF1-α are the key protective antigens against VL. Therefore, a fusion of T cell epitope rich domains of GP63, CPC and EF1-α was cloned, over expressed, purified and formulated in GLA-cationic liposome to overcome the major limitations of subunit vaccination strategy.

The routine and reliable method for diagnosis of VL is the microscopic enumeration of parasite nuclei from spleen, lymph nodes and liver aspirates (Ejazi, Bhattacharya et al. 2016) although an invasive and risky method. Rapid diagnostic immunoassays such as lateral flow based tests for detection of Leishmania specific antibodies have been employed commercially in many VL endemic areas. Recombinant antigens in rapid tests are preferred for their homogeneity and ease of preparation at industrial scale. Recombinant antigens such as rK39, rK28, rKE16 etc. (Sivakumar, Dey et al. 2008) (Abass, Bollig et al. 2013) (Pattabhi, Whittle et al. 2010) (Bhargava and Singh 2012) has been validated in VL diagnosis. (U.S. Pat. No. 5,912,166A, WO1994016331A). So far rK39 antigen in immunochromatographic strip form (InBios, Seattle, USA) is commercial available rapid test for VL especially in the Indian subcontinent. However its sensitivity did not match to the standard in Latin America, East Africa and Mediterranean countries (Ejazi, Bhattacharyya et al. 2018). None of the existing and upcoming antigens are being able to execute desired results. To achieve this unmet aim, a fusion of T cell epitope rich domains of GP63, CPC and EF1-α was used to determine the diagnostic potential in serum against human and canine leishmaniasis.

### OBJECTIVES OF THE INVENTION

The main objective of the present invention provides a liposomal formulation for treatment of visceral leishmaniasis. Another objective of the present invention is to provide a process of generation of fusion protein by combining the immunodominant epitope of key protective antigens CPC, GP63 and EF1-α.

Yet another objective of the present invention is to provide a novel fusion recombinant antigenic candidate for the diagnosis of visceral leishmaniasis in human and canine.

Yet another objective of the present invention is to provide a formulation of combination of fusion protein formulated in GLA-cationic liposome as a prospective strategy to overcome the major limitations of subunit vaccination strategy.

Another objective of the present invention is to provide a novel fusion recombinant antigenic candidate for the immunogenicity against visceral leishmaniasis in human.

### SUMMARY OF THE INVENTION

Accordingly, the present invention provides a vaccine and diagnostic potential of multiantigenic fusion protein against visceral leishmaniasis liposomal formulation for treatment of visceral leishmaniasis. It also relates to a fusion protein by combining the immunodominant epitope of key protective antigens CPC, GP63 and EF1-α. Another aspect of the present invention is to develop a method to clone, express and purify fusion protein which confers 559 amino acid sequence and a molecular weight of 61.9 kDa. In an embodiment of the present invention, it relates to the preparation of the novel formulation of combination of fusion protein in GLA-cationic liposome as a prospective vaccination strategy. Yet in another embodiment of the present invention, provided a novel fusion recombinant antigenic candidate for the immunogenicity against experimental visceral leishmaniasis. Yet in another embodiment of the present invention, provided a novel fusion recombinant antigenic candidate for the diagnosis of visceral leishmaniasis in human and canine using serum and urine samples. Yet in another embodiment of the present invention, it relates to the use of ELISA and Western blot to test the diagnostic potential of the fusion recombinant protein. Yet in another embodiment of the present invention, is to check the immunogenicity of the vaccine formulation in hamsters as disease model. Additional advantages of the present invention will be evident from the detailed description of the invention. The objects and embodiment of the present invention described as well as those inherent therein are not intended as limitations on the scope of the invention.

In one of the embodiment, the present invention discloses a liposomal formulation comprising of a fusion protein having SEQ ID No. 1

In another embodiment, the present invention discloses the liposomal formulation, wherein the fusion protein comprising a protein of Leishmania donovani comprising:
(a) glycoprotein 63 (GP63)
(b) elongation factor (EF1-α)
(c) cysteine protease C (CPC)

In yet another embodiment of the present invention discloses the liposomal formulation, wherein the liposomal formulation further comprising a cationic lipid, at least two neutral lipid and TLR agonists.

In other embodiment of the present invention discloses the liposomal formulation, wherein the cationic lipid is stearylamine (SA).

In other embodiment of the present invention discloses the liposomal formulation, wherein the neutral lipid are selected from distearylphosphatidylcholine (DSPC) and neutral lipid is selected from cholesterol or combination thereof.

In another embodiment of the present invention discloses the liposomal formulation, wherein the TLR agonists is glucopyranosyl lipid A (GLA)

The liposomal formulation as claimed in claim 3 comprising:
a) distearylphosphatidylcholine (DSPC);
b) stearylamine (SA);
c) cholesterol; and
d) glucopyranosyl lipid A (GLA)

In yet another embodiment of the present invention discloses a process for preparation of the liposomal formulation, wherein the process comprising the steps of:
(a) providing the fusion protein;
(b) cloning the fusion protein obtained in step (a) into a vector;
(c) dissolving a neutral lipid, a cationic lipid, a second neutral lipid and a TLR agonist in an organic solvent to obtain a solution followed by removing solvent using evaporator to obtain a thin lipid film;
(d) desiccating the thin lipid film as obtained in step (c) for a period of 14 to 16 hours to obtain dried lipid film;
(e) dispersing the dried lipid film as obtained in step (d) in either phosphate buffer saline alone or in the fusion protein of step (a) to obtain a mixture
(f) vortexing the mixture as obtained in step (e) and sonicating for a period in the range of 30 to 45 sec at 4° C. followed by keeping the mixture on ice for 2 h to stabilize the formed liposomes,
(g) removing the unentrapped proteins from the liposome formed in step (f) by ultracentrifugation to obtain a liposomal formulation.

In another embodiment of the present invention discloses the process, wherein the organic solvent is chloroform.

In other embodiment of the present invention discloses a pharmaceutical composition comprising a therapeutically effective amount of the liposomal formulation in a pharmaceutically acceptable carrier.

In yet another embodiment of the present invention discloses the liposomal formulation, wherein first neutral lipid, second neutral lipid, cationic lipid, TLR agonist and antigen is having the molar ratio of 7:2:2:0.0015:0.0026 in 0.02M PBS.

In another embodiment of the present invention discloses a kit for diagnosis of Leishmania infection comprising:
a) liposomal formulation of fusion protein,
b) an antibody conjugated with group of selected fluorophore, enzymes to detect the antigen-antibody complex indicating Leishmania infection.

In another embodiment of the present invention discloses a method of treating visceral leishmaniasis in a subject comprising administering to a said subject a therapeutic effective amount of liposomal formulation of fusion protein or a pharmaceutical composition.

In yet another embodiment of the present invention discloses the liposomal formulation of fusion protein for use in the treatment of visceral leishmaniasis in dogs and humans.

### BRIEF DESCRIPTION OF THE DRAWINGS

FIG. 1. Cloning of triple fusion epitope selected of fusion protein. (A) Cloning strategy of epitope of CPC, GP63 and EF1-α in fusion protein. (B) Confirmation of cloning through double digestion as shown in DNA gel electrophoresis.

FIG. 2. Identification of three recombinant antigens GP63, EF1-α, CPC and fusion protein through western blot. GP63, EF1α, CPC and their fusion were identified by their respective antibodies, anti-GP63 (A), anti-EF1α (B), and anti-CPC (C), corresponding to molecular weights 63 kDa, 51 kDa, and 36 kDa, respectively along with 61.8 kDa fusion protein.

FIG. 3. Lympho-proliferative and cytokine responses in PBMCs of immunized hamster. (A) Representative histogram showing percent proliferation, calculated in the indicated region, (B) The bar graphs show mean percent proliferation of the lymophocytes, which is inversely proportional to cell divisions, (C) Fold change in mRNA expression profiles of IL-4; TNF-α; IFN-γ.

FIG. 4. DTH response in BALB/c (A and B) and golden Syrian hamsters (C and D). Animals were immunized subcutaneously three times with 15 days interval with liposomal GP63, liposomal EF1α and liposomal CPC (A and B) and liposomal FUSION (C and D). Control groups received only PBS. DTH response was measured 10 days after the last immunization dose (A and C). Ten days after the last immunization they were challenged with 2.5×10⁷ freshly transformed stationary phase promastigotes of L. donovani and antigen specific DTH responses was measured again 3 months post infection (B and D). DTH response was measured as the difference (in mm) between the thickness of the test (2.5 μg/ml Ag-injected) and control (PBS-injected) footpads after 24 h. Results are shown as the mean±S.D. of three animals per group. Asterisks over the lines indicate significant differences between groups (*, p<0.05; **, p<0.01; ***, p<0.001; ****, P<0.0001).

FIG. 5. Evaluation of protection through Leishman Donovan units (LDU) against VL post-infection with L. donovani. BALB/c mice and hamsters were immunized subcutaneously three times with 15 days interval with liposomal GP63, liposomal EF1α and liposomal CPC (A and B) and liposomal FUSION (C and D), respectively. Ten days after the last immunization they were challenged with 2.5×10⁷ freshly transformed stationary phase promastigotes of L. donovani. Parasite burdens were measured 3 months post infection as Leishman Donovan Units (LDU), in liver (A and C) and spleen (C and D). The results are mean±SE of three individual animals per group. Asterisks over the lines indicate significant differences between groups (**, p<0.01; ***, p<0.001).

FIG. 6. Evaluation of protection through limiting dilution assay (LDA) against VL post-infection with L. donovani. BALB/c mice and hamsters were immunized subcutaneously three times with 15 days interval with liposomal GP63, liposomal EF1α and liposomal CPC (A and B) and liposomal FUSION (C and D), respectively. Control groups received only PBS. Ten days after the last immunization they were challenged with 2.5×10⁷ freshly transformed stationary phase promastigotes of L. donovani. Estimates of viable parasites in liver (A and C) and spleen (C and D) were determined by Limiting Dilution assay (LDA). The results are mean±SE of three individual animals per group. Asterisks over the lines indicate significant differences between groups (*,p<0.1; **, p<0.01; ***, p<0.001).

FIG. 7. Th1/Th2 cytokine profiles of immunized hamsters post-infection by quantitative realtime-PCR. Fold change in mRNA expression profiles of A, IFN-γ; B, TNF-α; C, IL-12; D, IL-4; E, TGF-b; F, IL-10; G, IL-2.

FIG. 8. Post-infection Th1 cytokine analysis. Splenocytes from vaccinated and control BALB/c mice (A and B) after infection challenge were stimulated with antigens and 72 h later concentrations of released, IL-12 (A) and IFN-γ (B) cytokines in the culture supernatants were quantitated by ELISA. Each sample was examined in duplicate. Results are shown as the mean±S.D. of three individual mice per group for post-infection. Asterisks over the lines indicate significant differences between groups (*, p<0.05; **, p<0.01; ***, p<0.001). Fold change in mRNA expression profiles of IL-12 (C) and IFN-γ (D). Each gene was normalized to the housekeeping gene (Hypoxanthine-guanine phosphoribosyltransferase, HGPRT) to avoid variations between different samples. The results are mean±SE of four individual hamsters per group. Asterisks over the lines indicate significant differences between groups (**, p<0.01; ***, p<0.001) as assessed by one-way ANOVA and Tukey's multiple comparison test.

FIG. 9. Post-infection Th2 cytokine analysis. Splenocytes from vaccinated and control BALB/c mice (A and B) after infection challenge were stimulated with antigens and 72 h later concentrations of released, IL-4 (A) and IL-10 (B) cytokines in the culture supernatants were quantitated by ELISA. Each sample was examined in duplicate. Results are shown as the mean±S.D. of three individual mice per group for post-infection. Asterisks over the lines indicate significant differences between groups (*, p<0.05; **, p<0.01; ***, p<0.001). Fold change in mRNA expression profiles of IL-4 (C) and IL-10 (D). Each gene was normalized to the housekeeping gene (Hypoxanthine-guanine phosphoribosyltransferase, HGPRT) to avoid variations between different samples. The results are mean±SE of four individual hamsters per group. Asterisks over the lines indicate significant differences between groups (**, p<0.01; ***, p<0.001) as assessed by one-way ANOVA and Tukey's multiple comparison test.

FIG. 10. Serum based immunoblot assay against fusion protein.

FIG. 11. Reactivity of fusion protein using serum and urine samples of human and canine leishmaniasis.

(A) Serum based reactivity of fusion protein using Indian serum samples; Visceral leishmaniasis (VL), n=83; Non-endemic Healthy control (NEHC), n=23; Endemic Healthy Control (EHC), n=14; Other diseases (OD), n=18, Follow-ups; n=15. Cut-off value 0.3275. Sensitivity (100%), specificity (95.65%)

(B) Serum based reactivity of fusion protein using human VL serum samples from Brazil were tested with active VL, n=33 and healthy control, HC, n=15. Cut-off value 0.25. sensitivity (84.85%), specificity (93.33%).

(C) Serum based reactivity of fusion protein using canine VL serum samples from Brazil were tested with active VL, n=39 and healthy control, HC, n=23. Cut-off value 0.38. Sensitivity (92.31%), specificity (82.61%).

(D) Reactivity of fusion protein using urine samples from Indian VL patients, n=34; EHC, n=7, OD n=11, NEHC n=14, FU n=6. Cut-off value 0.23. Sensitivity (100%), specificity (92.86%).

Statistical analysis was done using Graphpad prism Software version 8. Mann Whitney test was performed for unpaired samples and P value (P=<0.05) was considered to be significant.

FIG. 12. Colloidal gold based lateral flow test using serum (A) and urine samples (B).

### ABBREVIATIONS

VL: Visceral leishmaniasis
CL: Cutaneous leishmaniasis
ML: Mucocutaneous leishmaniasis
PKDL: Post kala-azar dermal leishmaniasis
ELISA: Enzyme Linked Immuno Sorbent Assay
PCR: Polymerase Chain Reaction
PVC: Poly Vinyl Chloride
SDS-PAGE: Sodium Dodecyl Sulfate-Polyacrylamide Gel Electrophoresis
EHC: Endemic Healthy Control
NEHC: Non Endemic Healthy Control
OD: Other Diseases
HC: Healthy Control
PBS: Phosphate Buffer Saline
GP63: Glycoprotein 63
EF1-α: Elongation factor 1 alpha
CPC: Cysteine protease C

### DETAILED DESCRIPTION OF THE INVENTION

The present invention provides a liposomal formulation for the treatment and diagnosis of visceral leishmaniasis in human and canine. It also relates to a fusion protein by combining the immunodominant epitope of key protective antigens CPC, GP63 and EF1-α.

The present invention further relates to the preparation of the novel formulation of combination of fusion protein in GLA-cationic liposome as a prospective vaccination strategy. The main aim of the present invention is to check the immunogenicity of the vaccine formulation in hamsters as disease model.

The present invention provides a pharmaceutical composition comprising a liposomal formulation and a pharmaceutically acceptable carrier.

The present invention further provides a kit for diagnosis of Leishmania infection wherein the kit comprising
a) liposomal formulation of fusion protein,
b) an antibody conjugated with group of selected fluorophore, enzymes to detect the antigen-antibody complex indicating Leishmania infection.

### EXAMPLES

The following examples are given by way of illustration of the present invention and therefore should not be construed to limit the scope of the present invention

### Example 1: Sera Collection and Preparation

Serum and urine samples of visceral leishmaniasis (VL) patients, before and after six months treatment (FU), endemic healthy controls (EHC), and Other diseases (OD), used in this study at the Indian Institute of Chemical Biology (IICB), Kolkata were earlier collected from the School of Tropical Medicine, Kolkata, West Bengal, and Rajendra Memorial Research Institute of Medical Sciences, Patna, Bihar with proper ethical clearance by the relevant hospitals and IICB. Nonendemic healthy controls (NEHC) were collected at the IICB. Sera from Brazilian VL patients and healthy controls along with infected and healthy canine sera were collected from Universidade Federal do Piaui, Teresina, Brazil.

### Example 2: Animals and Parasite

For parasite maintenance the L. donovani strain AG83 (ATCC PRA-413), originally isolated from an Indian kala-azar patient was periodically injected in appropriate numbers in 4-6 weeks old Syrian golden hamsters and reared in pathogen-free animal care facility of the Indian Institute of Chemical Biology. The infected hamsters were sacrificed at two months' infection to isolate L. donovani amastigotes from the spleens. These amastigotes were transformed into promastigotes in M199 supplemented with 10% FCS, 2 mM glutamine, penicillin G (100 U/ml) and streptomycin sulfate (100 μg/ml) at 22° C.

Studies were performed on 4-6 weeks old Syrian golden hamsters (Mesocricetus auratus) reared in pathogen-free animal care facility of CSIR-IICB, Kolkata. BALB/c mice, bred in the animal house facility of Indian Institute of Chemical Biology (Kolkata, India), were used for the antibody generation.

### Example 3; Antigen Cloning and Purification

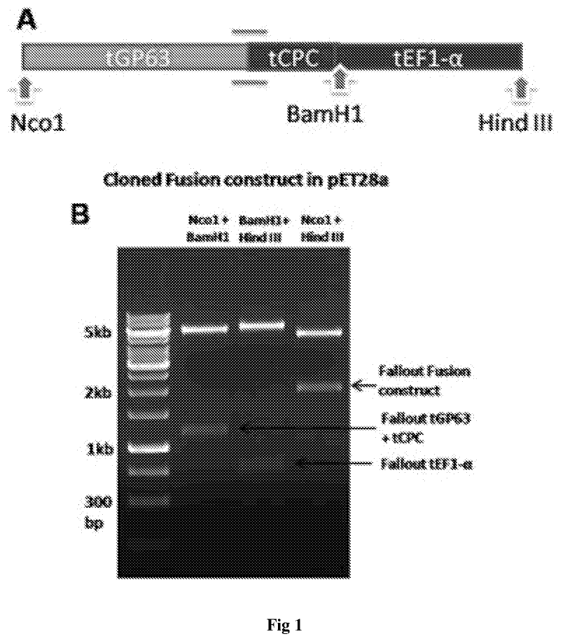

Whole gene sequence of the GP63, CPC and EF1-α from L. donovani strainAG83 (ATCC® PRA-413™) were analyzed for translational amino acid sequence. The prediction of immunogenicity of the different domains of the antigen was carried out by IEDB.org: Free epitope database and prediction resource, (https://www.iedb.org/). Prediction of epitope were restricted to 15 mer MHC-II and 9 mer MHC-I binding epitopes and overall MHC-I immunogenicity. In order to construct an open reading frame to express the domains of the three antigens as fusion protein, restriction site Nco1, BamH1 and HindIII were selected as these sites were absent in the DNA sequence of the corresponding three domains. Fusion of GP63 (amino acid 1 to 297) with CPC (amino acid 111 to 209) was designed with overlap PCR and then cloning at the Nco1 and BamH1 site of pET28a. The double fusion chimeric cloned plasmid was considered as a vector for insertion of the DNA sequence corresponding to EF1-α (amino acid 52 to 300) at the BamH1 and HindIII restriction site. The open reading frame was maintained to express the three domains together with a 6 Histidine tag for purification. The chimeric antigen was successfully cloned as depicted in FIG. 1.

Further the DNA sequence of fusion antigen was codon optimised to increase the yield of the protein and was subcloned in pET30a in between Ndel and Hind III.

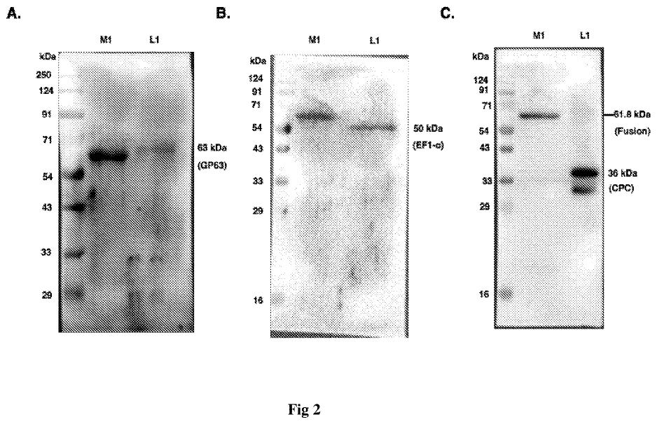

Plasmid isolated from positive clones was transformed in competent Rosetta strain of E. coli as described previously and plated in 40 μg/ml chloramphenicol and 50 μg/ml kanamycin LB agar plate. Log phase bacterial cultures were induced with IPTG (0.5 mM) and allowed to grow at 30° C. for another 4 hrs before harvesting the bacterial pellet by centrifugation at 6000 g for 10 mins. Pellets of the induced bacteria were resuspended and treated with lysozyme (1 mg/ml) in TBS for 30 minutes. Bacterial cells were then sonicated and centrifuged to collect the inclusion bodies as pellet and the supernatant. Inclusion bodies were solubilised in binding buffer composed of containing 8M Urea, 10 mM imidazole in 25 mM TBS and then centrifuged at 11,000 rpm for 30 mins to collect the supernatants that were added to pre-equilibrated Ni-NTA agarose matrix to allow His tagged proteins to bind. The bound matrix was washed 2 times with 50 mM imidazole containing urea buffer with 0.1% TritonX100. The washing process was repeated twice without TritonX100 in the washing buffer. Finally the bound recombinants were eluted using 500 mM imidazole containing elution buffer. The purified proteins were dialyzed in TBS buffer with decreasing urea concentration and finally exchanged with TBS/PBS buffer in 10-kDa Amicon cutoff membrane before analyzing the purity of the fusion protein by SDS-PAGE. The integrity and molecular weights of the purified fusion in comparison with the all the three constituent proteins were confirmed with SDS-PAGE as shown in FIG. 2. The proteins were identified through western blotting. The corresponding molecular weights of GP63, EF1α, CPC and fusion protein were found to be 63 kDA, 51 kDa, 36 kDa, and 61.8 kDa respectively.

### Example 4: Entrapment of Recombinant Fusion Protein in Liposomal Formulation

Liposomes containing recombinant antigens were prepared by the method described as follows. Initially the solution of lipid was prepared by dissolving 20 mg 1, 2-distearoyl-sn-glycero-3-phosphocholine (DSPC), 3 mg cholesterol, 2 mg SA (molar ratio of DSPC:Cholesterol:SA is 7:2:2) and 10 μg of GLA in methanolic-chloroform in a round bottomed flasks (REMCO). The lipid mixture was dissolved in methanolic-chloroform at 65° C. over a water bath to ensure complete solvation of the lipid so that no lipid particle sticks to the sides of the flask followed by evaporating the organic solvents to form a thin film in a round bottom glass flask using rotary evaporator. The lipid film was then desiccated in a vacuum desiccator for almost 16 hours. The film were then dispersed in either 1 ml of 0.02 M phosphate buffered saline (PBS) alone or containing 1 mg/ml of recombinant fusion protein making the molar ratio of DSPC:Cholesterol:SA:TLR agonist:antigen as 7:2:2:0.0015:0.0026. The mixture was then vortexed in the presence of glass beads and the suspension was sonicated for 1 min by an ultrasound probe sonicator (Takashi) thrice with 30 secs gap in between at 4° C. It was then kept on ice for 2 h to stabilize the formed liposomes. The unentrapped proteins were separated by three successive washings in PBS with ultracentrifugation at 100000 g for 1 h at 4° C. The protein entrapped in liposome was estimated by Lowry's method in the presence of 10% SDS and appropriate blanks using BSA as the standard protein Integrity after liposomal encapsulation was also evaluated by 12% SDS-PAGE followed by Coomassie staining and the measurement of entrapped proteins was used to calculate the entrapment efficiency of liposomes.

### Example 5: Immunization, DTH, proliferation of PBMCs and Splenocytes

For studies of fusion protein of GP63, EF1α and CPC, Syrian golden hamsters were immunized subcutaneously thrice with GLA DSPC-cationic liposomal formulation of the recombinant fusion. The study was performed on 4-6 weeks old Syrian golden hamsters. Initially, the hamsters were segregated into 4 groups with 5 hamsters per group. The hamsters were immunized subcutaneously between scapulae at back, three times at an interval of 14 days with 100 μl only PBS (G1), empty liposome (G2), 2.5 μg of rFusion (G3), 2.5 μg liposomal fusion (G4) in a total volume of 100 μl/animal/dose respectively. Ten days after the last booster dose DTH was done and all the hamsters were challenged intracardially with 2.5×10⁷ freshly transformed stationary phase promastigotes of L. donovani in 200 μl PBS. Immunological assays were carried out 3 months post infection.

For studies with individual proteins GP63, EF1α and CPC, BALB/c mice were immunised and rest of methodology remains same.

### DTH

Delayed type hypersensitivity (DTH) was determined 10 days post last vaccination and 3 months post infection in BALB/c and Syrian golden hamsters. The response was evaluated at 24 h by measuring the difference in swelling between two hind footpads, left footpad injected with purified 2.5 μg recombinant antigen per animal in a total volume of 50 μl PBS from that of the control (only PBS-injected) into right footpad with a constant pressure caliper.

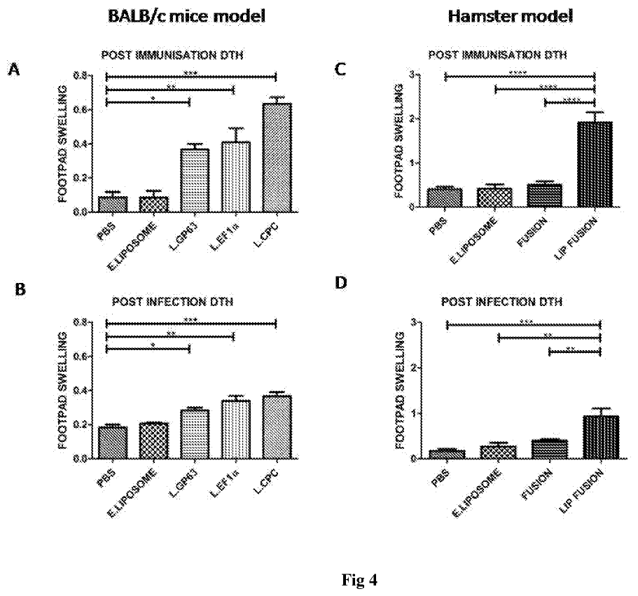

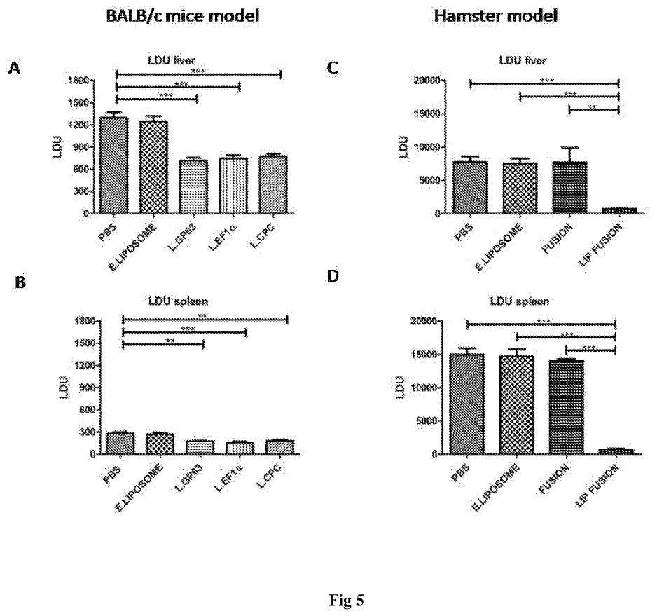

DTH measurement corresponds as an index of in vivo stimulation of recall cell-mediated immunity (CMI) towards leishmanial antigens determined 10 days after the last vaccination dose (4A and 4C). Immunization with liposomal fusion of recombinant antigens i.e. LIP FUSION induced the highest level of DTH response (p<0.0001) (FIG. 4B) compared to the other vaccinated groups where liposomal CPC vaccination induced the maximum DTH (p<0.001) followed by L.EF1α (p<0.01) and L.GP63 (p<0.05) respectively (FIG. 4A). Immunization with fusion of recombinant antigens i.e. LIP FUSION maintained the highest level of DTH even after infectious challenge (p<0.001) (FIG. 4D) in comparison with single antigen groups (FIG. 4C).

### Proliferation of PBMCs of Hamsters

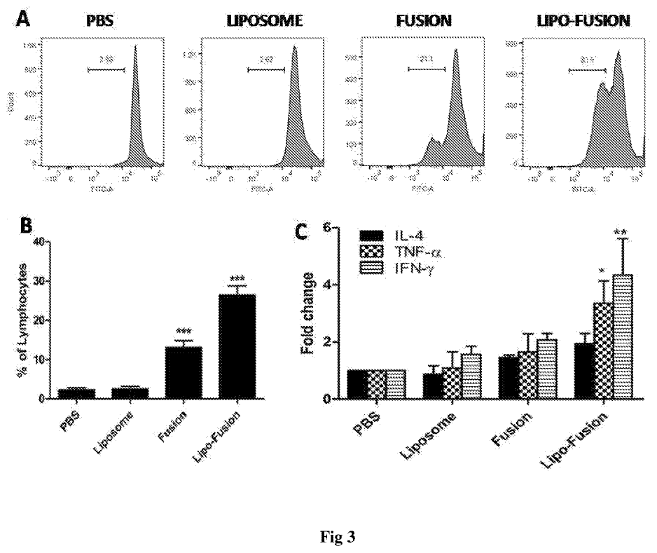

For proliferation, the PBMCs of hamsters were prestained with CFSE (Carboxyfluorescein succinimidyl ester) before antigen stimulation and after 48 hrs, the fluorescence of the PBMCs of hamsters were determined by flow cytometry as reported previously. Briefly 106/ml PBMCs in FBS free media were labelled with 1 μl CFSE at a final concentration of 5 μM. Cells were then incubated for 20 minutes at room temperature or 37° C., protected from light. Culture medium (containing at least 1% protein) was added five times the original staining volume of the cells and incubated for 5 minutes at room temperature. This step removes any free dye remaining in the solution. The cells were centrifuged at 900 g for 5 minutes. The cells were then plated at 10⁵ cells/well in a 24 well plate and stimulated as per experiment. Hamsters immunized with liposomal fusion protein showed significant proliferative response in the post immunisation PBMCs (FIG. 3B) indicating persistence of CMI response.

### Example 6: LDU

Infection was evaluated by estimation of parasite burden in the liver and spleen of BALB/c and Syrian golden hamsters in terms of Leishman Donovan units, three months post challenge. Giemsa stained smears of liver and spleen were microscopically observed to count the nuclei of parasites inside the cells of tissues. LDU was calculated as number of parasite nuclei in 1000 nucleated cells per organ weight of the tissue smears.

In order to correlate the vaccination induced immuno-modulation with the protective efficacy, parasite burden in the liver (FIGS. 5A and 5C) and spleen (FIGS. 5B and 5D) of the challenge infected BALB/c mice and hamsters were determined. The giemsa stained parasite nuclei from the organ smears of mice (FIGS. 5A and 5B) and hamsters (FIGS. 5C and 5D) challenged with virulent L. donovani were microscopically counted to determine the level of infectivity. Liposomal FUSION vaccinated hamsters showed 85% and 93% reduction in parasite load in liver (FIG. 5C) and spleen (FIG. 5D), respectively in comparison with control group whereas other vaccinated groups, liposomal GP63, liposomal EF1α and liposomal CPC showed 46% and 50% reduction in parasite load in liver and spleen, respectively (FIGS. 5A and 5B).

### Limiting Dilution Assay (LDA)

To estimate live parasites in the infected liver and spleen, limiting dilution assay (LDA) was employed. For LDA, 1 mg homogenized infected tissues were fivefold serially diluted in Schneider's media containing 20% FBS and cultured for 3 weeks at 22° C. days in 96-well culture plates for allowing transformation of promastigotes. Presence of promastigotes was observed in each well. The reciprocal of the highest dilution of tissues that led to transformation of promastigotes multiplied by the total organ weight gave the estimate of viable parasite burden in the organs by LDA.

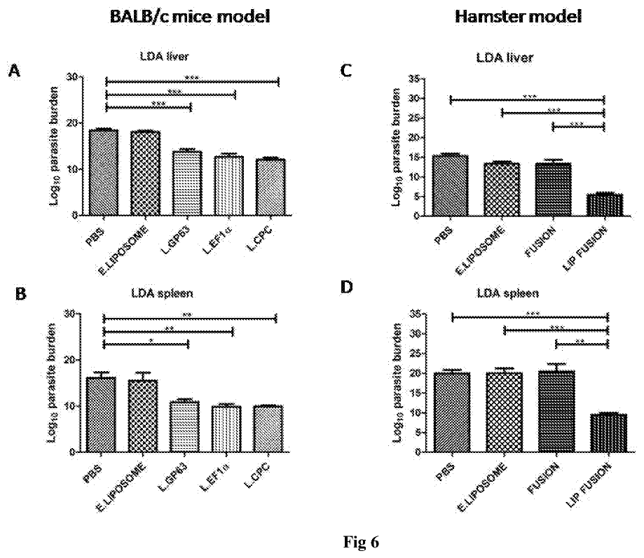

The outcome of infection with L. donovani leads to cumulative multiplication of parasites in both liver and spleen leading to hepatosplenomegaly and weight loss. Immunization with Liposomal FUSION (FIGS. 6C and 6D) demonstrated an 8 to 9 fold reduction in parasite burden in liver (FIG. 6C) and 9 to 10 fold reduction in parasite burden in spleen (FIG. 6D) when compared to the other vaccinated groups such as liposomal GP63 (6 and 5 folds), liposomal EF1α (7 and 5 folds) and liposomal CPC (8 and 4 folds) in the liver and spleen, respectively (FIGS. 6A and 6B). These data, therefore demonstrate that Liposomal FUSION antigen can induce better protection against Leishmania infection in the susceptible infection model (hamster) compared to liposomal GP63, liposomal EF1α or liposomal CPC alone.

### Example 7: Cytokine Assay at mRNA Level

Liver and spleen of immunized hamsters after infection were collected and stored in trizol. The organs were mashed into single suspension using hand homogeniser. The total mRNA was isolated by Trizol method. Briefly, 1 ml of single cell suspension was pelleted down at 10,000 g for 10 min at 4° C. The supernatant was collected and 200 μl chloroform was added. The mixture was vortexed and the suspension was incubated at room temperature for 20 min. The solution was centrifuged at 11,000 rpm for 20 min at 4° C. The aqueous phase was transferred carefully and mixed with 2.5× isopropanol and incubated for 15 min at room temperature. The mixture was centrifuged at 11,000 rpm for 15 min. The supernatant was decanted carefully followed by washing the pellet with 100% ethanol. The ethanolic solution was centrifuged 11,000 rpm for 20 min and RNA pellet was air dried. The pellet was dissolved in ultrapure DEPC-water and the RNA concentration was measured in Nanodrop 2000 (Thermo Scientific, USA). Total cDNA library of each group was constructed using Evoscript universal cDNA master (Roche). Briefly 2.5 μg of RNA, 2 μl of reverse transcriptase, 4 μl of reaction buffer was used per reaction of 20 μl. cDNA was synthesized by incubating the tubes for 15 min at 42° C., 5 min at 85° C., 15 min at 65° C. After the reaction was over cDNA was stored at −20° C.

Levels of IFN-γ, TNF-α, TGF-β, IL-2, IL-4, IL-12 and IL-10 specific mRNA were quantified by Real Time PCR (Roche). Briefly measurement of levels of cytokine was carried out using 1 μl of hamster, 300 nM cytokine specific primers and 5 μl of SYBR Green Real-Time PCR Master Mix (thermo) were used for a 10 μl reaction mixture. Hamster HGPRT (Hypoxanthine-guanine phosphoribosyltransferase) was used as an endogenous control. Real time PCR was carried out at an initial incubation for 10 minutes at 95° C. and then 40 cycles of 95° C. for 15 sec and 58° C. for 30 sec, and 72° C. for 30 sec in light cycler 96 (Roche). Each measurement was carried out in triplicate. The relative expression of mRNA was calculated by the comparative Ct (2^{-ΔΔCt}) method in terms of fold change normalized to HGPRT expression. No template control was done to rule out any nonspecific reactions. The fold change in the levels of IFN-γ, TNF-α and IL-4 in the immunized groups were compared with the PBS control groups. The fold change in the mRNA transcripts of cytokines, indicate a significant increase in the levels of IFN-γ and TNF-α from the stimulated PBMCs liposomal vaccinated hamsters. The increase in IL-4 transcripts in the vaccinated hamsters was not found to be significant.

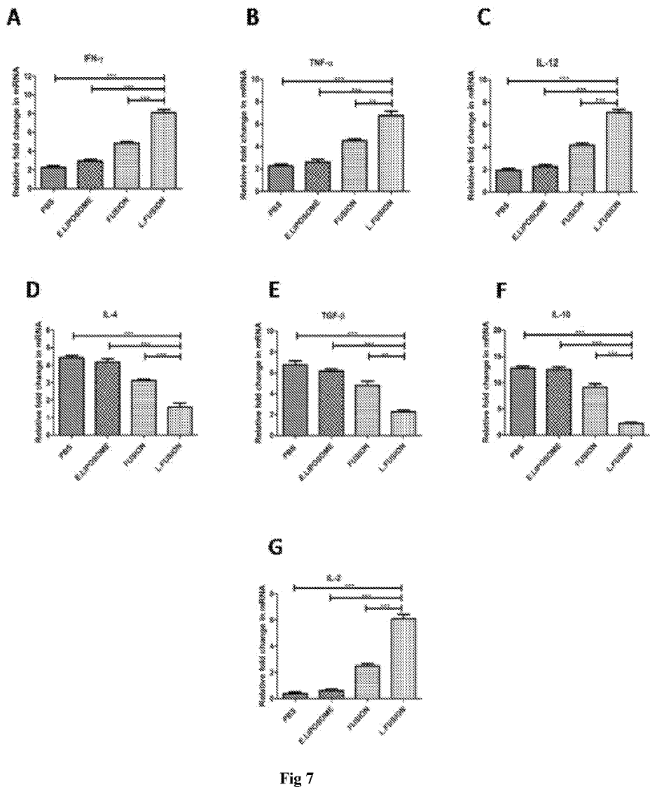

The post infection fold change in the levels of A, IFN-γ; B, TNF-α; C, IL-12; D, IL-4; E, TGF-b; F, IL-10; G, IL-2. in the immunized groups were compared with the other control groups. Levels of IFN-γ, TNF-α, IL-12 and IL-2 increased in the group immunised with liposomal fusion and level of TGF-β, IL-10 and IL-4 fall significantly in the in the immunized groups were compared with the PBS control groups (FIG. 7).

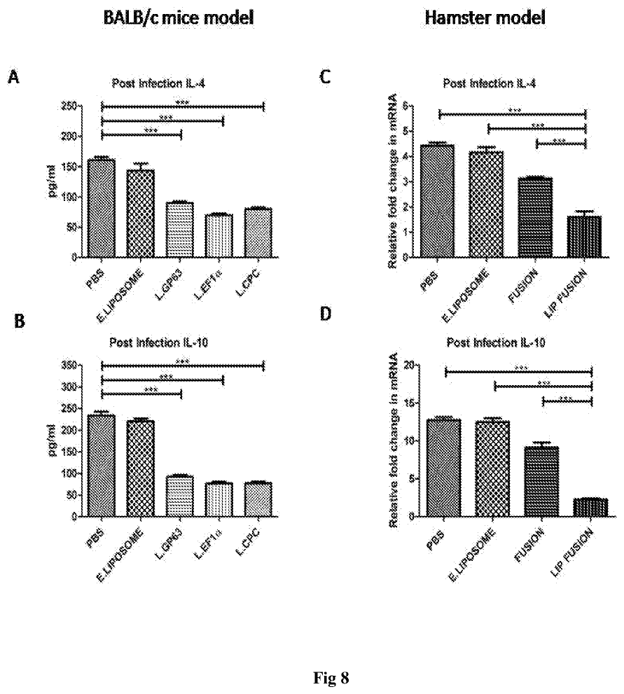

The level of cytokine expression in BALB/c mice was determined through ELISA (FIGS. 8A and 8B) and the level of cytokine expression in hamsters was estimated by real time PCR using HGPRT as reference control (FIGS. 8C and 8D). Levels of IL-12 (FIG. 8C) and IFN-γ (FIG. 8D) corresponding to Th1 increased significantly (p<0.001) in the group immunised with liposomal FUSION (FIGS. 8C and 8D) in comparison with control group. The levels IL-12 and IFN-γ in other vaccinated groups such as liposomal GP63, liposomal EF1α and liposomal CPC (FIGS. 8A and 8B) also increased significantly (p<0.001) in comparison with PBS control groups.

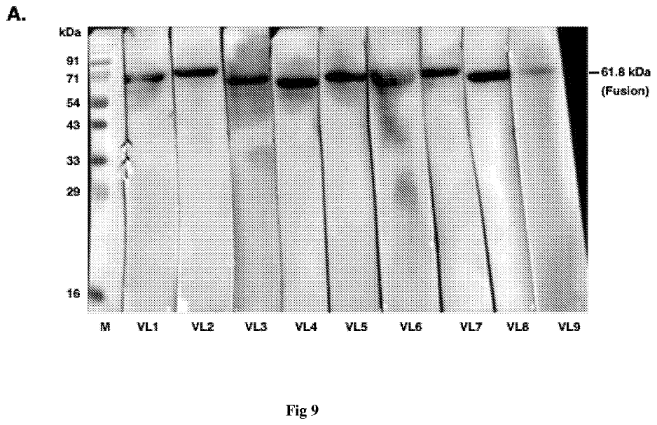

Levels of IL-4 (FIG. 9C) and IL-10 (FIG. 9D) corresponding to Th2 decreased significantly (p<0.001) in the group immunised with liposomal FUSION (FIGS. 9C and 9D) in comparison with control group. The levels IL-4 and IL-10 in other vaccinated groups such as liposomal GP63, liposomal EF1α and liposomal CPC (FIGS. 9A and 9B) also decreased significantly (p<0.001) in comparison with PBS control groups. However a direct compare between the two models cannot be done as the cytokines measured in mice are secreted proteins whereas in the hamsters they are at the mRNA levels.

### Example 8: Immunoblot Assay

Fusion protein was run in the SDS-PAGE and transferred electrophoretically onto nitrocellulose membrane using Trans-Blot Turbo (BioRad) transferred system under semidried condition for seven minutes using 1.5 mA and 25 Volts. Transferred protein on membrane was visualized when blot was dipped into Ponceau dye. Each lane from the blot was cut and Ponceau dye was completely destained. Membrane was blocked with 5% bovine serum albumin (BSA) for 1 hour at 37° C. After washing the membrane was incubated with diluted Indian serum samples (1:1000) overnight at 4° C. in 2.5% BSA in PBS. Next day strips were further washed with 100 mM TBS & 0.05% Tween-20 and incubated with peroxidase-conjugated goat anti-human IgG (1:4000 dilutions). After washing strips were placed in proper order and developed using substrate luminol and H₂O₂ and pictures were taken in ChemiDoc chemiluminescent apparatus (BioRad Laboratories, USA).

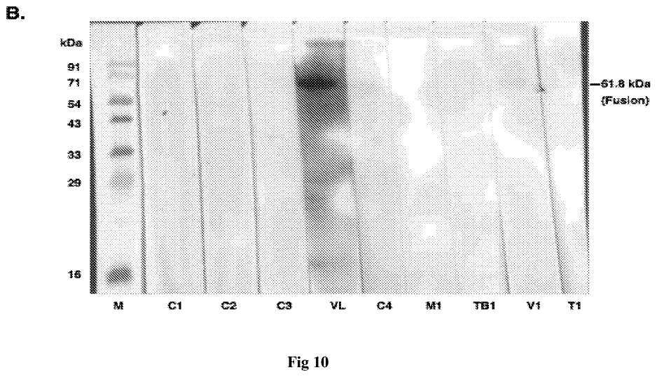

Total nine VL samples were tested in the immunoblot assay using fusion protein. All the active VL serum samples recognized the fusion protein at 61.8 kDa (FIG. 10A). Control serum samples containing, two nonendemic healthy controls, two endemic controls, one of each malaria, tuberculosis, viral fever, and typhoid however did not show any cross reactivity with the fusion protein (FIG. 10B).

### Example 9: ELISA

Capture antibody ELISA was done by coating 1 μg/well of fusion protein with phosphate buffer in 96 well microtitre plates and incubated overnight at 4° C. Unbound antigen was washed using phosphate buffer saline (PBS) with 0.05% tween-20 detergent and blocked with bovine serum albumin (1% BSA) in PBS for 2 h at 37° C. Further, sample carrying antibodies were added at a dilution of 1:1000 times for serum followed by 1:5000 diluted HRP-conjugated anti-human IgG for 45 minutes at 37° C. After washing in between each steps, 100 μl/well substrate 3,3′,5,5′-Tetramethylbenzidine (TMB) were added for 5-10 minutes and reaction was stopped using 2N H₂SO₄. Finally, optical density was read at 492 nm. Cut-off values were set according to the ROC curve obtained using healthy controls.

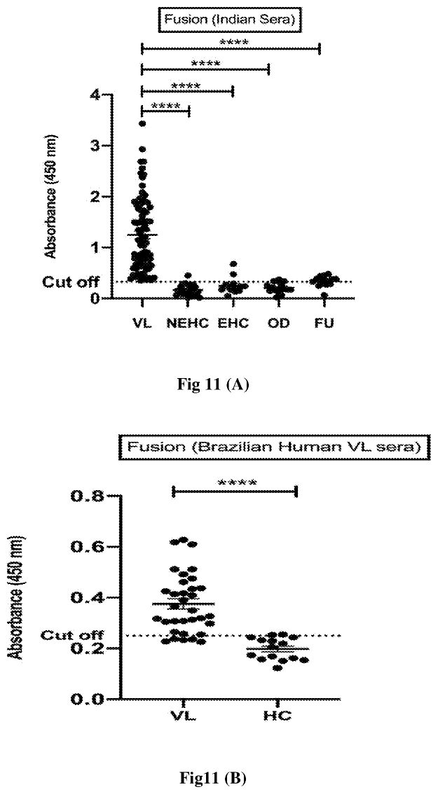

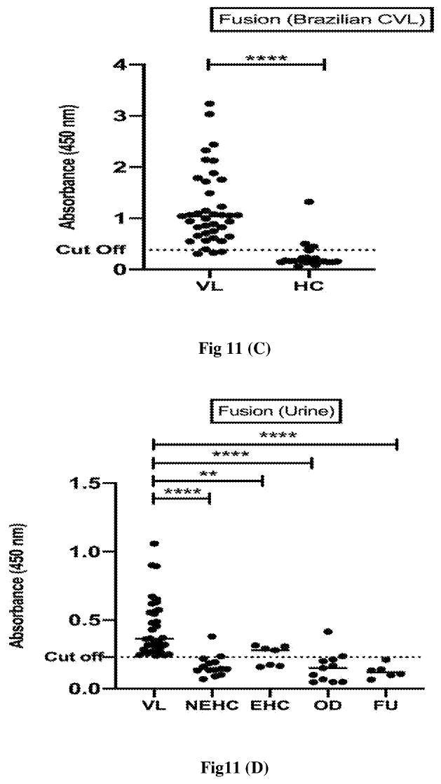

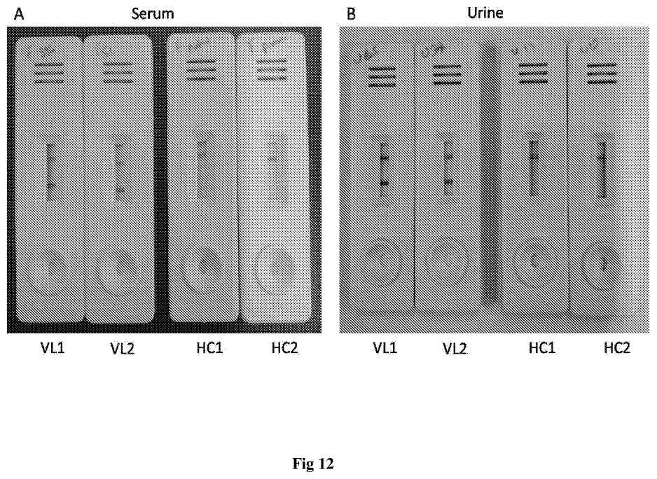

ELISA using human sera from Indian patients showed 100% sensitivity and 95.65% specificity at 0.3275 of cut-off (FIG. 11A). None of the samples from VL positive cases were found negative whereas only one healthy control showed cross reactivity with the fusion protein. 15 VL sera after 6 months treatment showed significant decline compared to VL with the fusion protein. ELISA with Brazilian sera depicted 84.85% sensitivity and 93.33% specificity at a cut-off value of 0.25. Out of 33 sera used from active VL cases five showed false negative result. However, one healthy control sample was false positive (FIG. 11B). The sensitivity obtained in ELISA using canine sera from Brazil demonstrated 92.31% sensitivity and 82.61% specificity at a cut-off value of 0.38 (FIG. 11C). Antibody titer in urine samples through ELISA demonstrated 100% sensitivity and 92.86% specificity with healthy controls at a cut-off value of 0.23 (FIG. 11D). 6 VL urine sample after 6 months treatment showed no reactivity with the fusion protein.

### Example 10: Lateral Flow Assay Using Fusion Protein

Backed nitrocellulose membrane (0.45 μm, mdi membranes) was used to coat the fusion protein (1 μg/ml) and antimouse antibodies (1 μg/ml) at the test and control lines, respectively, using autodispenser (Precore Biosystems). Coated membranes were then dried in 37° C. for 30 min. 0.5 ml thick conjugate pad was dispensed with Protein G conjugated colloidal gold (Ubio) in 10% sucrose PBS buffer at 1:1 dilution and dried at 37° C. for 30 min. Dried conjugate pad was pasted at the test line side with a overlap of 2 mm with the nitrocellulose membrane. Subsequently substrate pad was used with a overlap of 2 mm with the conjugate pad. Absorbent pad was used at the control line side in the downstream. Assembled membrane was then cut into 2 mm thick strips and fixed in the cassette and stored for the assay under desiccation. For chase buffer 20 mM Tris buffer saline+0.05% Tween 20 and 0.01% sodium azide was optimised. For the lateral flow assay, 10 μl of serum was applied at the sample buffer side followed by 25 μl of chase buffer. Result became visible as colored bands and interpreted within 5 min.

In principle antibodies present in the VL infected samples migrate from sample pad to conjugate pad and bind with the protein G of the conjugate. Chase buffer gives the fluidity to the complex. This complex then moves towards nitrocellulose and binds with the L. donovani fusion protein at the test line. Antibody-conjugate complex moves and binds to the control line. For non VL cases antibodies specific to fusion protein was not present in the sample thus no reaction took place at the test line (FIG. 12A). However, protein G-gold conjugate always bind with the antimouse antibodies at the control line. In total, 10 confirmed VL cases and 7 non endemic healthy control sera samples were used for the LFT study. All the positive VL samples showed clear test and control line bands suggesting 100% sensitivity of the test. 100% specificity was found with healthy controls. LFT with urine samples were also tested in LFT as depicted in FIG. 12B.

### Advantages of the Present Invention

The advantage of the present invention is to provide a good immunogenic vaccine formulation that can elicit T cell based immunity against VL along with a subcutaneous delivery system.
The unique feature about the formulation is that being a recombinant protein it can match with the safety of subunit vaccine. Not only in terms of safety but being a multi epitopic formulation even its efficacy is comparable with that of live attenuated vaccine.
Moreover, the delivery vehicle comprising of liposomes and adjuvant further increased the immunogenicity and stability of the recombinant fusion protein.
The invention also provides a good diagnostic candidate. Most of the test available for VL diagnosis is serological and their performance vary in different endemic areas.
In contrast the present invention provides good sensitivity and specificity and is used with serum and urine samples of both human and dogs. Being a multiantigenic recombinant protein, chances of detecting VL positive samples increases many fold.

### REFERENCES

Abass, E., N. Bollig, et al. (2013). “rKLO8, a novel Leishmania donovani-derived recombinant immunodominant protein for sensitive detection of visceral leishmaniasis in Sudan.” PLOS Negl Trop Dis 7 (7): e2322.
Bhargava, P. and R. Singh (2012). “Developments in diagnosis and antileishmanial drugs.” Interdiscip Perspect Infect Dis 2012:626838.
Bhowmick, S., R. Ravindran, et al. (2007). “Leishmanial antigens in liposomes promote protective immunity and provide immunotherapy against visceral leishmaniasis via polarized Th1 response.” Vaccine 25 (35): 6544-6556.
Bhowmick, S., R. Ravindran, et al. (2008). “GP63 in stable cationic liposomes confers sustained vaccine immunity to susceptible BALB/c mice infected with Leishmania donovani.” Infect Immun 76 (3): 1003-1015.
Bi, K., Y. Chen, et al. (2018). “Current Visceral Leishmaniasis Research: A Research Review to Inspire Future Study.” Biomed Res Int 2018:9872095.
Das, A. and N. Ali (2012). “Vaccine Development Against Leishmania donovani.” Front Immunol 3:99.
Didwania, N., M. Shadab, et al. (2017). “Alternative to Chemotherapy—The Unmet Demand against Leishmaniasis.” Front Immunol 8:1779.
Ejazi, S. A., P. Bhattacharya, et al. (2016). “Noninvasive Diagnosis of Visceral Leishmaniasis: Development and Evaluation of Two Urine-Based Immunoassays for Detection of Leishmania donovani Infection in India.” PLOS Negl Trop Dis 10 (10): e0005035.
Ejazi, S. A., A. Bhattacharyya, et al. (2018). “Immunoproteomic Identification and Characterization of Leishmania Membrane Proteins as Non-Invasive Diagnostic Candidates for Clinical Visceral Leishmaniasis.” Sci Rep 8 (1): 12110.
Ismail, N., A. Kaul, et al. (2017). “Immunization with Live Attenuated Leishmania donovani Centrin (−/−) Parasites Is Efficacious in Asymptomatic Infection.” Front Immunol 8:1788.
Mazumder, S., M. Maji, et al. (2011). “Potentiating effects of MPL on DSPC bearing cationic liposomes promote recombinant GP63 vaccine efficacy: high immunogenicity and protection.” PLOS Negl Trop Dis 5 (12): e1429.
Pattabhi, S., J. Whittle, et al. (2010). “Design, development and evaluation of rK28-based point-of-care tests for improving rapid diagnosis of visceral leishmaniasis.” PLOS Negl Trop Dis 4 (9).
Sabur, A., S. Bhowmick, et al. (2018). “Liposomal Elongation Factor-1 alpha Triggers Effector CD4 and CD8 T Cells for Induction of Long-Lasting Protective Immunity against Visceral Leishmaniasis.” Front Immunol 9:18.
Sivakumar, R., A. Dey, et al. (2008). “Expression and characterization of a recombinant kinesin antigen from an old Indian strain (DD8) of Leishmania donovani and comparing it with a commercially available antigen from a newly isolated (KE16) strain of L. donovani.” Infect Genet Evol 8 (3): 313-322.

1. A liposomal formulation comprising a fusion protein having SEQ ID No. 1.

2. The liposomal formulation as claimed in claim 1, wherein the fusion protein of Leishmania donovani comprises:
(a) glycoprotein 63 (GP63)
(b) elongation factor (EF1-α)
(c) cysteine protease C (CPC)

3. The liposomal formulation as claimed in claim 1, wherein the liposomal formulation further comprises a cationic lipid, at least two neutral lipids and TLR agonists.

4. The liposomal formulation as claimed in claim 3, wherein the cationic lipid is stearylamine (SA).

5. The liposomal formulation as claimed in claim 3, wherein the first neutral lipid is selected from distearylphosphatidylcholine (DSPC) and second neutral lipid is selected from cholesterol or combination thereof.

6. The liposomal formulation as claimed in claim 3, wherein the TLR agonists is glucopyranosyl lipid A (GLA).

7. The liposomal formulation as claimed in claim 3 comprising:
a) distearylphosphatidylcholine (DSPC);
b) stearylamine (SA);
c) cholesterol; and
d) glucopyranosyl lipid A (GLA)

8. A process for preparation of the liposomal formulation as claimed in claim 1 wherein the process comprises the steps of:
(a) collecting and preparing serum and urine samples;
(b) cloning and purification of a polynucleotide encoding the fusion protein as claimed in claim 1 into a vector to obtain a recombinant fusion protein;
(c) encapsulation of the recombinant fusion protein as obtained in step (b) to obtain the liposomal formulation as claimed in claim 1.

9. A pharmaceutical composition comprising a therapeutically effective amount of the liposomal formulation as claimed in claim 1 in a pharmaceutically acceptable carrier.

10. The liposomal formulation as claimed in claim 1, wherein the first neutral lipid, second neutral lipid, cationic lipid, TLR agonist and antigen have a molar ratio of 7:2:2:0.0015:0.0026 in 0.02M PBS.

11. A kit for diagnosis of Leishmania infection comprising:
(a) fusion protein as claimed in claim 1; and
(b) an antibody conjugated with fluorophore, enzymes to detect an antigen-antibody complex indicating Leishmania infection.

12. A method of treating visceral leishmaniasis in a subject comprising administering to said subject a therapeutic effective amount of liposomal formulation of fusion protein of claim 1.

13. The liposomal formulation of fusion protein as claimed in claim 1, for use in the treatment of visceral leishmaniasis in humans and dogs.

14. A method of treating visceral leishmaniasis in a subject comprising administering to said subject a therapeutic effective amount of liposomal formulation of a pharmaceutical composition of claim 9.
